(19) United States
(12) Patent Application Publication
(10) Pub. No.: US 2022/0214292 A1
(43) Pub. Date: Jul. 7, 2022

(54) PORTABLE NMR INSTRUMENTATION AND METHODS FOR ANALYSIS OF BODY FLUIDS

(71) Applicant: The Regents of the University of California, Oakland, CA (US)

(72) Inventors: Matthew P. AUGUSTINE, Oakland, CA (US); John MADSEN, Oakland, CA (US); Johnny PHAN, Oakland, CA (US); Joseph POURTABIB, Oakland, CA (US); Sophia Noelle FRICKE, Oakland, CA (US); Shahab CHIZARI, Oakland, CA (US); Nam K. TRAN, Oakland, CA (US)

(21) Appl. No.: 17/600,901

(22) PCT Filed: Apr. 6, 2020
(86) PCT No.: PCT/US2020/026857

§ 371 Date: Oct. 1, 2021

Related U.S. Application Data
(60) Provisional application No. 62/830,291, filed on Apr. 5, 2019.

(51) Int. Cl. G01N 24/08; G01N 33/49; G01R 33/44

(52) U.S. Cl. CPC G01N 24/082; G01R 33/448; G01N 33/49

(57) ABSTRACT

Methods and instrumentation for determining the water content of a body fluid such as blood plasma by portable nuclear magnetic resonance (NMR) relaxometry are provided.

### CROSS-REFERENCE TO RELATED APPLICATIONS

The present application claims priority to U.S. Provisional Application No. 62/830,291 filed Apr. 5, 2019, the full disclosure of which is incorporated by reference in its entirety for all purposes

### STATEMENT AS TO RIGHTS TO INVENTIONS MADE UNDER FEDERALLY SPONSORED RESEARCH AND DEVELOPMENT

This invention was made with government support under Grant No. R25EB012963 awarded by the National Institutes of Health. The government has certain rights in the invention.

### BACKGROUND

A large percentage of medical decisions are based on laboratory tests, many of which are blood chemistry assays. Because sixty percent of the human body is water, which serves as an important constituent for many biochemical pathways and homeostatic processes, the human plasma water content (PWC) influences many fundamental medical blood chemistry tests like those used for measuring electrolyte and metabolite concentrations (Lyon & Baskin, 34 Lab. Med. 357 (2003); Straseski et al., 57 Clin. Chem. 1566 (2011); Fogh-Andersen, Wimberley, Thode, & Siggard-Andersen, 189 Clin. Chim. Acta 33 (1990); Fogh-Andersen & D'Orazio, 44 Clin. Chem. 655 (1998); Nguyen, Ornekian, Butch, & Kurtz 292 Am. J. Physiol. Renal. Physiol. F1652 (2007)). However, despite the dependence of blood test accuracy on blood water content, the routine clinical measurement of the amount of this simple molecule in various organs, muscles, and bodily fluids has remained elusive. Although gravimetric methods involving sample lyophilization have been used to determine PWC (Waugh, 18 Metabolism 706 (1969)), they are not practical in a high throughput clinical laboratory where speed is critical for patient care. This is because the current standard lyophilization method for measuring PWC is a time-intensive process that requires about 24 hours. In this process, blood samples are evaporated and weighed to determine the amount of water they originally contained. The delays from this procedure are prohibitive in a clinical laboratory that must process thousands of samples daily, and are unacceptable in situations that require urgent treatment decisions. Estimates of PWC have also been accomplished by accounting for all of the non-water protein and lipid sample components and by comparing electrolyte concentrations obtained with direct and indirect ion selective electrodes (ISEs). These approaches are less accurate than the gravimetric approach and also consume the patient sample. Due to these limitations, medical practitioners are forced to make generalized assumptions for PWC, which can result in substantial risk to patient care.

The development and clinical implementation of both ISE and substrate specific electrodes (SSEs) for respective electrolyte and metabolite measurements revolutionized clinical chemistry in the 1980s. Early ISEs were “indirect ISEs” (I-ISEs) that required pre-analytical dilution to achieve sufficient volume to cover the sensor electrodes. Unfortunately, I-ISE specimen dilution assumed a normal PWC of 93%. It was quickly recognized that this PWC assumption was false since samples containing excess protein and/or lipids create a water exclusion effect that significantly alters the PWC (Lyon & Baskin, 34 Lab. Med. 357 (2003); Nguyen, Ornekian, Butch, & Kurtz 292 Am. J. Physiol. Renal. Physiol. F1652 (2007); Lopez, Burtis, Ashwood, & Bruns (eds), Tietz Textbook of Clinical Chemistry and Molecular Diagnosis 2238 (2012)). Since the I-ISE dilution volume is unchanged, the exclusion effect introduces an additional dilution that falsely lowers measured electrolyte concentrations. Today, I-ISEs continue to be used in mainframe laboratory analyzers due to their longevity and cost-effectiveness, while direct ISEs (D-ISEs) not requiring pre-analytical dilution have been developed for point-of-care applications.

In contrast to ISEs, SSEs measure the molality of metabolites such as glucose and creatinine. As before, it was assumed that PWC remained unchanged. Fogh-Andersen et al. in the early 1990's proposed a whole blood-to-plasma glucose conversion factor of 1.11 based on the 93% PWC assumption (Fogh-Andersen, Wimberley, Thode, & Siggard-Andersen, 189 Clin. Chim. Acta 33 (1990); Fogh-Andersen & D'Orazio, 44 Clin. Chem. 655 (1998)). This conversion factor was adopted by the International Federation for Clinical Chemistry (IFCC) in 2008 (D'Orazio, 51 Clin. Chem. 1573 (2005)). However, subsequent studies showed that whole blood glucose and creatinine measurements change during critical illness where PWC may also significantly change (Straseski et al., 57 Clin. Chem. 1566 (2011); Lyon, DuBois, Fick, & Lyon, 4 J. Diabetes Sci. Technol. 1479 (2010)). Variance in PWC between patients can influence many test results, with blood electrolyte and metabolite measurements being perhaps the most notable. The need therefore exists for methods providing a rapid and accurate measurement of PWC. The present disclosure provides these and other needs.

### BRIEF SUMMARY

Provided herein are methods and equipment for the use of portable nuclear magnetic resonance (NMR) relaxometry in the rapid determination of blood plasma water content (PWC), in the clinic as well as other settings such as portable bloodbanks, field hospitals, and ambulances. The NMR-based methods described herein accurately correlate PWC to relaxometry measurements (e.g., T₂ and T₁ decay constants) in plasma samples. Rapid testing methods as provided herein for measurement of PWC can allow clinicians to improve the accuracy of blood chemistry assays and diagnostic tests, improving patient care and reducing waste. For example, there is a great deal of clinical interest in using PWC to analyze burn patient hydration status. Blood transfusions that drastically vary in PWC from the patient could cause shock. The NMR-based methods of the present disclosure have been applied to the analysis of animal plasma samples and have achieved a 98.7% PWC prediction accuracy, which matches the ˜98% accuracy of the current standard (and more time-intensive) lyophilization-based technique. The PWC obtained according to the disclosure herein can be used, for example, to correct sodium cation concentrations reported from direct ion-selective electrode tests. The accuracy of PWC determination with the provided methods and apparatus is comparable to that of the gravimetric method that requires sample lyophilization. The rapid turnaround time, non-destructive nature, and portable footprint of the provided measurement methods can improve treatment outcomes by, for example, better enabling rapid, point-of-care clinical electrolyte estimates.

In one aspect, the disclosure is to a method for determining the water content of a body fluid. The method includes analyzing a body fluid sample by portable NMR relaxometry. In some embodiments, the analyzing includes determining a spin-spin relaxation constant T_{2,sample} of the body fluid sample and/or a spin-lattice rate constant T_{1,sample} of the body fluid sample, and calculating the water content of the body fluid sample using the determined T_{2,sample} and/or T_{1,sample}. In some embodiments, the calculating includes applying a correlation between water content of the body fluid and one or both of T₂ and T₁, e.g., T_{2,sample} and T_{1,sample}. In some embodiments, the correlation is derived from a measurement of spin-spin relaxation constants of each of one or more, e.g. two or more, standard solutions, and/or spin-lattice rate constants of each of the one or more, e.g., two or more, standard solutions.

In another aspect, the disclosure is to a method for correcting an electrolyte concentration estimate. The method includes estimating an electrolyte concentration in a sample using, as a non-limiting example, an ion selective electrode. The method further includes determining the water content of the sample using any of the water content determination methods disclosed herein. The method further includes correcting the estimated electrolyte concentration using the determined water content.

In another aspect, the disclosure is to a portable NMR apparatus for the analysis of water content in body fluids. In some embodiments, the NMR apparatus includes a tank circuit probe having a solenoid radiofrequency coil configured to accept a body fluid sample container, wherein the tank circuit probe is disposed between two sides of an opposing poleface magnet. These and other aspects, objects and embodiments will become more apparent with the detailed disclosure and figures that follow

### BRIEF DESCRIPTION OF THE DRAWINGS

FIG. 1 is a graph showing an example of the raw, transient relaxation data generated by applying the CPMG pulse sequence to the PWC=84% pseudoplasma sample. The shaded area corresponds to the 1,600 slightly overlapping spin echoes observed during the 68-s-long experiment.

FIG. 2 is a graph showing the transient yielded by application of a Fourier transform based filter to the data in FIG. 1.

FIG. 3 is a graph showing an example of the raw, transient relaxation data generated by applying the saturation recovery pulse sequence to the PWC=84% pseudoplasma sample.

FIG. 4 is a graph showing the transient yielded by application of a Fourier transform based filter to the data in FIG. 3.

FIG. 5 is a graph showing the correlation of gravimetric PWC with NMR-determined T₂ value for the pseudoplasma and human lyophilized plasma sample sets as solid squares and diamonds respectively. The solid and dashed lines for the respective pseudoplasma and human lyophilized plasma samples were calculated from the appropriate A and B values in Table 3. The error bars largely obscured by the data markers indicate 95% confidence.

FIG. 6 is a graph showing the correlation of gravimetric PWC with NMR-determined T₁ value for the pseudoplasma and human lyophilized plasma sample sets as solid squares and diamonds respectively. The solid and dashed lines for the respective pseudoplasma and human lyophilized plasma samples were calculated from the appropriate A and B values in Table 3. The error bars indicate 95% confidence.

FIG. 7 shows an example of a process for analysis of the PWC of blood plasma according to the present disclosure.

FIG. 8 shows an example of a process for analysis of the PWC of blood plasma according to the present disclosure.

FIG. 9 shows an example of portable NMR instrumentation for analysis of body fluids according to the present disclosure.

FIG. 10 shows an example of portable NMR instrumentation for analysis of body fluids according to the present disclosure.

### DETAILED DESCRIPTION

Plasma water content (PWC) affects the accuracy of routine laboratory measurements. Altered PWC in vivo is also attributed to disease. Until this disclosure, the routine measurement of PWC in clinical specimens was not feasible. Although PWC is a relevant metric for many medical diagnostic procedures, the routine clinical measurement of PWC has remained elusive. The inventors have now discovered that particular methods and apparatus using portable nuclear magnetic resonance (NMR) allow for the nondestructive and quick measurement of PWC without altering the specimen. More specifically, the inventors have developed NMR spectroscopy methods and apparatus to quickly and inexpensively detect the water content of whole blood and serum, based on the surprising observation that the two NMR relaxation time constants T₂ and T₁ correlate with water content or PWC at low magnetic field.

In brief, NMR is a powerful analytical technique that can non-invasively probe samples with unorthodox geometries and differentiate between components of chemical mixtures (Blumich, Perlo, & Casanova, 52 Prog. Nucl. Magn. Reson. Spectrosc. 197 (2008)). When placed in a static external magnetic field, B₀, a sample like water will magnetize. The size of magnetization is related to the nuclear spins in the proton nuclei, ¹H, residing in the hydrogen atoms. This magnetization is typically measured by applying a pulsed radio frequency (RF) magnetic field, B₁, directed perpendicular to B₀ at the Larmor frequency, a value that depends on the size of B₀ and the structure of the ¹H nucleus (Freeman, A Handbook of Nuclear Magnetic Resonance (1997); Levitt, Spin Dynamics: Basics of Nuclear Magnetic Resonance (2001)). In the Examples disclosed herein, B₀=0.367 T and the Larmor frequency used for the RF pulses is 15.63 MHz. The time constant for a sample to magnetize when placed in a magnet is called T₁, while the time constant for the signal to decay to zero, or equivalently, the time constant for magnetization created perpendicular to B₀ with an RF pulse to decay to zero is T₂.

NMR relaxometry has already been successfully applied to the study of blood plasma (Cistola & Robinson, 83 Trends Anal. Chem. 53 (2016)). It is well known that the dominant proton NMR signal in blood plasma is attributed to water, because water generally accounts for >80% of blood and >90% of blood plasma or serum by mass (Id.). Spin relaxation occurs predominantly through dipolar coupling brought about by locally fluctuating magnetic fields (Freeman, A Handbook of Nuclear Magnetic Resonance (1997); Levitt, Spin Dynamics: Basics of Nuclear Magnetic Resonance (2001)). The chief effector of these field fluctuations is the Brownian movement of molecules (Einstein, Investigations on the Theory of the Brownian Movement (1956)). In addition to water, a myriad of proteins, lipoproteins, and metabolites are also present in blood, and these molecules interact with water molecules via the formation hydrogen bonds. In turn, these interactions affect the spin relaxation properties of water by altering the rotational correlation time of the bound-state water, which is inversely proportional to T₂ and T₁, and is defined as the time required for a molecule or molecular complex to rotate by one radian. Since the correlation time increases with an increase in molecular size (i.e. in the addition of blood components to pure water), as well as with an increase in viscosity or decrease in temperature as described by the generalized Stokes-Einstein-Debye equation (Id.; Debye, Polar Molecules: The Chemical Catalog (1929)), plasma samples with lower water content will have faster relaxation times.

Moreover, rapid proton exchange occurs between free water, protein-bound water, and other hydrogen atoms on proteins (Hills, 76 Mol. Phys. 489 (1992)). Since the exchange time for these protons is short in comparison to T₂ and T₁, with respective timescales of 10⁻⁹ s for exchange versus 10⁻³-10⁰ s for spin relaxation, this phenomenon results in a weighted averaging of T₂ and T₁ values for bound and unbound water.

Over 90% of the total protein concentration in blood can be attributed to the following most abundant proteins, which are albumin, immunoglobins, transferrin, fibrinogen, α2-macroglobulin, α1-antitrypsin, C3 complement, and haptoglobin. Moreover, >80% of this total concentration corresponds to the first two respective proteins (Lundblad, 1 Internet J. Genom. Proteom. 1 (2003)). This means that, in effect, only a few different proteins have a significant influence on the nuclear spin relaxation of plasma water. The relaxation rate of plasma water therefore correlates linearly with the net concentration of proteins in the blood (Kang, Gore, & Armitage, 1 Magn. Reson. Med. 396 (1984); Raeymaekers, Borghys, & Eisendrath, 6 Magn. Reson. Med. 212 (1988); Schumacher et al., 13 Magn. Reson. Med. 103 (1990)).

The approach pioneered by Cistola and Robinson (83 Trends Anal. Chem. 53 (2016)) is to use the relaxation properties of water to monitor the type and relative quantities of blood proteins and lipoproteins. This provides information relating to global biomarkers that can be used as early indicators of disease. In contrast, the inventors have now developed the specific methods and apparatus disclosed herein to use a proton NMR signal to characterize the percentage of water present in a plasma sample.

The disclosed methods and apparatus provide several benefits not present with current procedures and instruments. Since NMR is non-destructive to the sample and testing can be accomplished in a matter of minutes, it is an ideal tool for the clinical laboratory. The accuracy of PWC determination with NMR using the disclosed methods is comparable to the gravimetric method that requires sample lyophilization. The rapid turnaround time and non-destructive nature of the NMR approach is a significant advantage in comparison to lyophilization to determine PWC. Given that it takes about one minute to run a Carr-Purcell-Meiboon-Gill (CPMG) experiment on a plasma sample, the delay will have a negligible effect on the throughput of a modern clinical laboratory. In fact, this time is comparable to the time required to perform a hemolysis index to evaluate specimen integrity. This provided methods and apparatus can thus be implemented immediately to run on all plasma samples intended to measure electrolytes and metabolites such as glucose. The NMR instrument can be configured to run automatically and does not disrupt the workflow in any foreseeable way.

Moreover, while most conventional PWC tests require a considerable volume of blood, the methods provided herein only requires a small amount of sample (ca. 1 mL) for NMR analysis. NMR is also non-destructive, meaning that the same sample can be used for other laboratory tests. Altogether, these advantages are helpful for patients because they allow the provided methods to reduce the amount of blood that has to be drawn, and yield faster and more accurate blood test results and diagnoses.

Furthermore, the low radio frequency (RF) magnetic fields required to perform NMR with permanent magnets can penetrate metal layers covering a sample. Recent technological advancements have made portable NMR spectroscopy economically and practically feasible. The ease with which portable NMR spectroscopy can be customized to address specific scientific and clinical problems makes it an extremely attractive technique. Nevertheless, there are consequences to working at the low magnetic fields common to portable NMR spectroscopy. Specifically, signals are weaker and have decreased resolution in comparison to traditional NMR. As a result, the transverse and longitudinal relaxation rate constants (T₂ and T₁) are the parameters typically measured at low field, rather than chemical shifts and J-couplings (Blumich, Perlo, & Casanova, 52 Prog. Nucl. Magn. Reson. Spectrosc. 197 (2008)).

The disclosed methods and apparatus use the analytical performance of low-field NMR relaxometry to obtain PWC measurements. The approach described herein takes advantage of a correlation between PWC and measured T₂ and T₁ values. By constructing models from the correlation of PWC to T₂ and T₁ values obtained from measurements on standard samples, the T₂ and T₁ values from similar substances like porcine and human blood can be used to predict an appropriate PWC value. The accuracy of the approach has been verified, e.g., using a contrived pseudoplasma matrix as well as porcine and model human blood samples. As described below, the NMR PWC measurement can further be used to correct clinical I-ISE sodium cation (Nat) concentration estimates.

The present disclosure therefore provides a nondestructive method to measure PWC quickly and accurately through NMR relaxometry. The methods and instruments provided herein can be used in the care of hospital patients (e.g., in the course of burn patient hydration in an intensive care unit), for monitoring transfusions and blood banks, for conducting coagulation studies, and for making clinical diagnoses (e.g., diagnosis of malaria). The instruments and methods can be automated by including multiple RF probes for multiple plasma samples, by integrating the instrumentation with clinical analyzers for instantaneous correction, by including slide probes to switch out samples, and/or by equipping the instrumentation with self-calibration modules.

In contrast to assays that detect the concentrations of individual metabolites and biomarkers directly, measurement of plasma T₂ and T₁ values can provide information about the bulk, macroscopic properties of a plasma sample. This type of analysis, in conjunction with parameters obtained from traditional laboratory testing, offers a way to monitor net changes in blood plasma that have the potential to inform clinicians about the overall health of a patient. The NMR-based methods presented herein benefit significantly from their simplicity. The measurement provided by the methods is not necessarily intended to be the sole technique implemented for clinical analysis of a sample, rather, it can be used together with the host of other laboratory techniques that provide accurate measurements of other blood components. Importantly, the speed with which this method provides results can be beneficial in time-sensitive cases, since PWC is not yet routinely measured, despite the fact that knowledge of PWC would better inform clinicians about a patient's health.

### EMBODIMENTS

The following embodiments are contemplated. All combinations of features and embodiment are contemplated.

### Embodiment 1

A method for determining the water content of a body fluid, the method comprising: analyzing a body fluid sample by portable nuclear magnetic resonance (NMR) relaxometry. Body fluids include, but are not limited to, whole blood, serum, plasma, urine, sputum, bronchial lavage fluid, tears, nipple aspirate, lymph, saliva, fine needle aspirate (FNA), cerebral spinal fluid, and combinations thereof.

### Embodiment 2

An embodiment of embodiment 1, wherein the analyzing of the body fluid sample by NMR relaxometry comprises: determining, using NMR relaxometry, a spin-spin relaxation rate constant T_{2,sample} of the body fluid sample and/or a spin-lattice rate constant T_{1,sample} of the body fluid sample; and calculating the water content of the body fluid sample using the determined T_{2,sample} and/or T_{1,sample}.

### Embodiment 3

An embodiment of embodiment 1 or 2, wherein the calculating comprises applying a correlation between water content of the body fluid and one or both of T₂ and T₁.

### Embodiment 4

An embodiment of embodiment 3, wherein the correlation is derived from a measurement of a spin-spin relaxation rate constant T_{2,standard} of a standard solution and/or a spin-lattice rate constant T_{1,standard} of the standard solution, wherein the standard solution has a known standard water content. A sample having an unknown water content (a test sample) can be determined from a standard curve made from standard solutions, such as 2, 3, 4, 5, 6, or more standard solutions. In some embodiments, a dilution series of a standard solution can be used to make a standard curve

### Embodiment 5

An embodiment of embodiment 3 or 4, wherein the correlation is derived from measurements of spin-spin relaxation rate constants of two or more standard solutions and/or spin-lattice rate constants of the two or more standard solutions, wherein at least two of the two or more standard solutions have different known standard water contents.

### Embodiment 6

An embodiment of embodiment 4 or 5, wherein each known standard water content is between 70% and 98%.

### Embodiment 7

An embodiment of any of the embodiments of embodiment 3-6, wherein the correlation comprises one or more log-linear functions.

### Embodiment 8

An embodiment of any of the embodiments of embodiment 3-7, wherein the correlation comprises one or more functions each independently having the form: (water content)=A log(Tₙ)+B, wherein A and B are each independently constants, and wherein n is 1 or 2. The values of the constants A and B can be derived using measurements of standard solutions, as is describe in the Example 2 derivation of the exemplary A and B values of Table 3. The variable Tₙ refers to NMR relaxation time constant values for spin-spin (when n=2) or spin-lattice (when n=1), which can be obtained as described in the Examples 1, 3, and 5 measurements of the exemplary T₁ and T₂ values of Tables 1, 2, 4, and 5.

### Embodiment 9

An embodiment of any of the embodiments of embodiment 4-8, wherein each known standard water content is determined using gravimetric data.

### Embodiment 10

An embodiment of any of the embodiments of embodiment 4-9, wherein at least one standard solution comprises bovine serum albumin, lipid, sodium chloride, and urea.

### Embodiment 11

An embodiment of any of the embodiments of embodiment 4-10, wherein at least one standard solution comprises porcine blood or plasma.

### Embodiment 12

An embodiment of any of the embodiments of embodiment 4-11, wherein at least one standard solution comprises human blood or plasma.

### Embodiment 13

An embodiment of any of the embodiments of embodiment 1-12, wherein the body fluid sample is a blood plasma sample, and wherein the water content of the body fluid sample is a plasma water content (PWC).

### Embodiment 14

An embodiment of any of the embodiments of embodiment 2-13, wherein each spin-lattice rate constant is measured by saturation recovery.

### Embodiment 15

An embodiment of any of the embodiments of embodiment 2-14, wherein each spin-spin relaxation rate constant and spin-lattice rate constant is determined further using single component exponential fitting with non-linear least squares regression.

### Embodiment 16

An embodiment of any of the embodiments of embodiment 2-15, wherein each spin-spin relaxation rate constants and spin-lattice rate constant is determined further using a Fourier transformation, a multiplication by a Gaussian peak, and an inverse Fourier transformation.

### Embodiment 17

A method for correcting an electrolyte concentration estimate, the method comprising: estimating an electrolyte concentration in a sample using an ion selective electrode; determining the water content of the sample using the method of an embodiment of any of the embodiments of embodiment 1-16; and correcting the estimated electrolyte concentration using the determined water content

### Embodiment 18

A method for determining the water content of a body fluid, the method comprising: analyzing a body fluid sample by portable nuclear magnetic resonance (NMR) relaxometry.

### Embodiment 19

An embodiment of embodiment 18, wherein the analyzing of the body fluid sample by NMR relaxometry comprises: determining a spin-spin relaxation rate constant T₂ and/or a spin-lattice rate constant T₁; and correlating T₂ and/or T₁ with the water content of the body fluid sample.

### Embodiment 20

An embodiment of embodiment 18 or 19, wherein the body fluid sample is a blood plasma sample.

### Embodiment 21

A portable nuclear magnetic resonance (NMR) apparatus for the analysis of water content in body fluids.

### Embodiment 22

An embodiment of embodiment 21, having a tank circuit probe comprising a solenoid radiofrequency (RF) coil configured to accept a body fluid sample container, wherein the tank circuit probe is disposed between two sides of an opposing poleface magnet.

### EXAMPLES

The present disclosure will be better understood in view of the following non-limiting examples. The following examples are intended for illustrative purposes only and do not limit in any way the scope of the present disclosure

### Example 1. Analysis of T₂ and T₁

To determine if NMR T₁ and T₂ values correlate with PWC, a 15-sample set of pseudoplasma was prepared with 70%<PWC<98%. Two separate liquids were used for standard materials and all chemicals were purchased from Sigma Aldrich, Lee Biosolutions, or similar vendors. The first standard referred to here as “pseudoplasma” was prepared by mixing bovine serum albumin, INTRALIPID®, sodium chloride, and urea with water to produce PWC percentages ranging from 70-98%, in increments of 2%. Normal saline water was used, since it is relatively (albeit not completely) isotonic to normal plasma. No special precautions were taken to de-oxygenate the water, in order to mimic the properties of clinical blood plasma samples and the fact that normal saline is dosed in the same manner in live patients. The partial pressure of oxygen (pO2) in these samples would be comparable to what is present at atmospheric pressure (˜160 mmHg). The pO2 was constant throughout all of the samples.

To better mimic real world samples, the second standard, referred to here as “human lyophilized plasma” was purchased from a commercial vendor and diluted in the same way as the first standard. The correlation of NMR and gravimetric data for the human lyophilized plasma sample set was used to estimate the PWC in a test sample set of commercially available porcine blood purchased from the UC Davis Meat Lab. The sample sets used in the Examples disclosed herein are blood plasma or designed to simulate blood plasma. Complications arising from the presence of paramagnetic deoxyhemoglobin in whole blood that dramatically shorten the spin relaxation times (Silvennoinen, Kettunen, & Clingman, 405 Arch. Biochem. Biophys. 78 (2002)) are avoided here, as the actual samples of interest are blood plasma, not whole blood.

Both NMR T₂ and T₁ values, in addition to gravimetric estimates of PWC, were obtained for each of these samples and are reported in Table 1. All ¹H NMR experiments at a 15.63 MHz Larmor frequency were performed at 0.367 T using a Model 4S SpinCore opposing pole face magnet. Free precession signals were obtained from a Tecmag Apollo LF1 spectrometer and ENI 250 kHz-110 MHz RF amplifier connected to a tuned solenoid coil wrapped around a 1.8-mL tube that holds the sample in the center of the magnet. Operation in this way typically yields an 8.5 μs, π/2 RF pulse with 56 W of applied RF power. The Carr-Purcell-Meiboom-Gill (CPMG) (Meiboom & Gill, 29 Rev. Sci. Instrum. 688 (1958)), spin-spin T₂ time constant value and the saturation recovery, spin-lattice T₁ time constant value were measured in triplicate at a controlled temperature of 23° C. for each sample.

For CPMG experiments used to measure T₂, the delay between π RF pulses was 3 ms, 1600 spin echoes were obtained, and the repetition time for signal averaging was 13 s. To cancel artifacts arising from pulse imperfections, the initial π/2 RF pulse and the receiver were cycled between +x and −x phase while holding the π RF pulse phase constant at +y. In all cases, signal averaging summed 12 CPMG transient signals. The effect of diffusion on T₂ measurements was inconsequential given the 3-ms π pulse spacing and the <0.5 G cm⁻¹ field gradient presented by the permanent magnet. Other procedural details on the pulse sequences and phase-sensitive detection were as described previously (Freeman, A Handbook of Nuclear Magnetic Resonance (1997); Levitt, Spin Dynamics: Basics of Nuclear Magnetic Resonance (2001); [11, 12, 24, 25].

For T₁ measurement, a saturation recovery experiment was preferred because it is faster than an inversion recovery pulse sequence (Freeman, A Handbook of Nuclear Magnetic Resonance (1997); Levitt, Spin Dynamics: Basics of Nuclear Magnetic Resonance (2001)). A comparison between the two pulse sequences yielded T₁ values within a few ms of each other for the entire range of pseudoplasma samples considered. As such, it was determined that the reduced sampling window of the saturation recovery experiment did not appreciably sacrifice measurement precision. The number of free induction decays recorded for the saturation recovery experiments was 80, the repetition time was 13 s, and no signal averaging was required.

The signal-to-noise of the raw NMR data was improved with a digital signal filter written in Matlab.

Gravimetric methods were as described previously (Waugh, 18 Metabolism 706 (1969)). Here a portion of each sample was weighed before and after lyophilization to determine the amount of water loss. All Na⁺ concentrations estimated from clinical lab I-ISEs were corrected by multiplication with a ratio of 93% to the PWC determined from the human lyophilized plasma NMR model shown in Table 1.

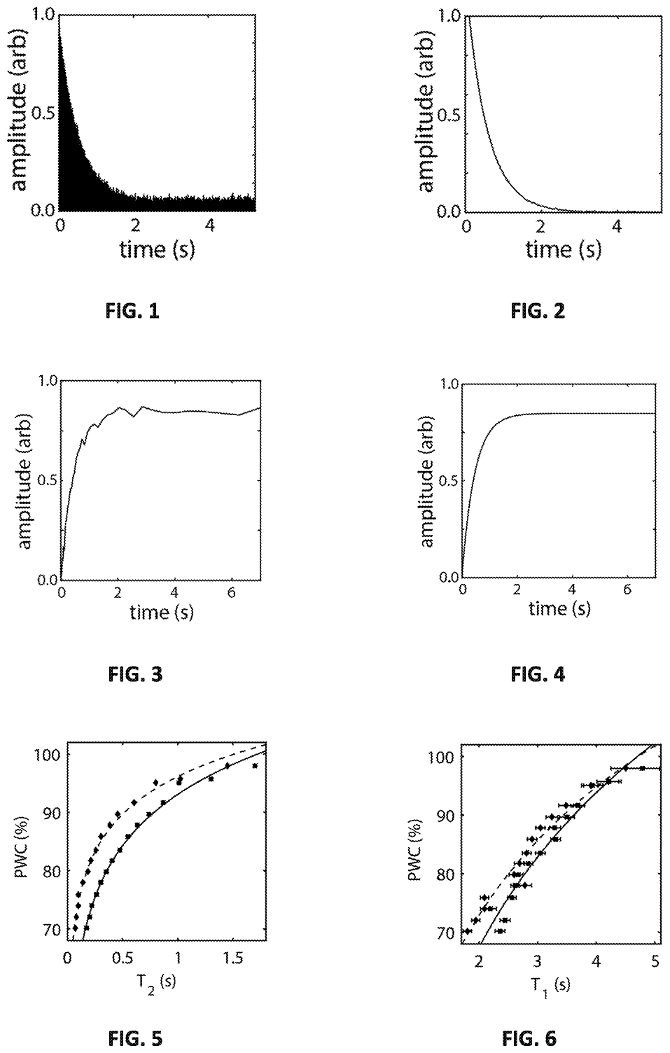

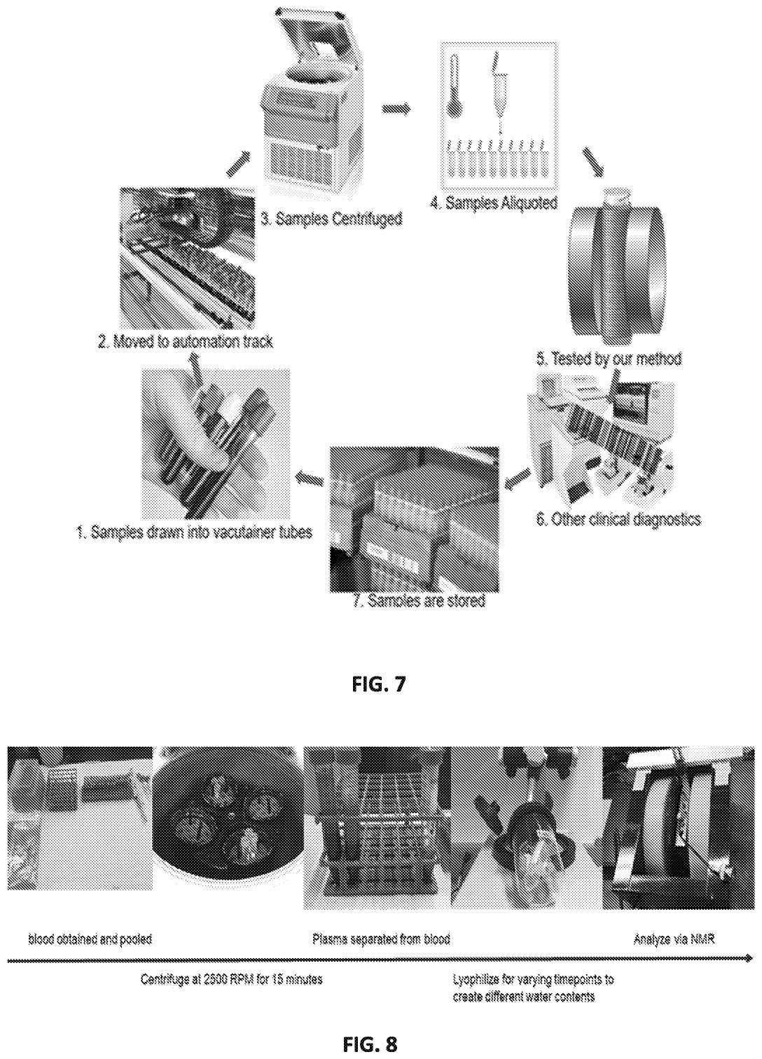

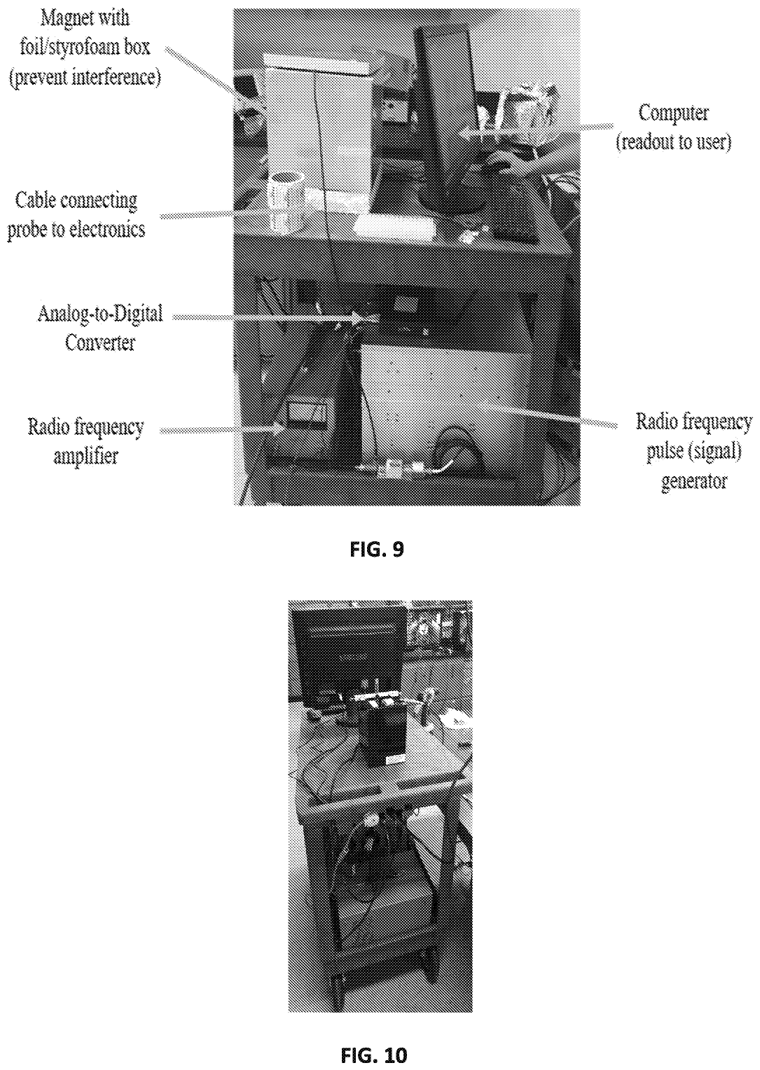

FIG. 1 shows the raw CPMG transient signal obtained from the PWC=84% pseudoplasma sample. The individual spin echoes that cause the shaded area beneath the exponential envelope disappear upon post data acquisition signal processing to yield the transient in FIG. 2. A similar improvement in signal-to-noise is obtained for the saturation recovery transient signal for the same sample as shown in FIGS. 3 and 4. Analysis of transient signals like those in FIGS. 1-4 for all of the pseudoplasma and human lyophilized plasma standards led to the T₂ and T₁ time constant values shown in the second and third columns in Tables 1 and 2 respectively. The PWC values obtained from gravimetric analysis of these same samples are shown in the fourth column of these tables.

TABLE 1
Summary of NMR and gravimetric data obtained
from the pseudoplasma sample set

| | NMR time | | | | | prediction | |
|---|---|---|---|---|---|---|---|
| sam- | constants (ms) | | PWC (%) | | | accuracy (%)^{a} | |
| ple | T₂^{b} | T₁^{c} | grav.^{d} | T₂ calc.^{d} | T₁ calc.^{d} | T₂^{e} | T₁^{f} |
| 1 | 172 | 2360 | 70.2 | 72.4 | 73.8 | 96.8 | 94.8 |
| 2 | 201 | 2444 | 72.0 | 74.2 | 75.1 | 97.0 | 95.8 |
| 3 | 220 | 2196 | 74.0 | 75.3 | 71.0 | 98.3 | 95.9 |
| 4 | 263 | 2561 | 75.9 | 77.3 | 76.9 | 98.2 | 98.7 |
| 5 | 302 | 2619 | 78.0 | 78.9 | 77.7 | 98.8 | 99.6 |
| 6 | 351 | 2671 | 79.8 | 80.7 | 78.5 | 98.9 | 98.4 |
| 7 | 404 | 2840 | 81.7 | 82.3 | 80.8 | 99.3 | 98.9 |
| 8 | 475 | 3046 | 83.5 | 84.2 | 83.5 | 99.2 | 100.0 |
| 9 | 547 | 3307 | 85.9 | 85.8 | 86.6 | 99.9 | 99.1 |
| 10 | 633 | 3292 | 87.8 | 87.5 | 86.4 | 99.6 | 98.4 |
| 11 | 740 | 3501 | 89.7 | 89.3 | 88.8 | 99.6 | 99.0 |
| 12 | 869 | 3686 | 91.7 | 91.2 | 90.7 | 99.5 | 99.0 |
| 13 | 1012 | 3930 | 95.1 | 93.0 | 93.2 | 97.8 | 98.0 |
| 14 | 1302 | 4218 | 95.7 | 95.9 | 95.9 | 99.8 | 99.8 |
| 15 | 1700 | 4788 | 98.0 | 99.0 | 100.7 | 99.0 | 97.2 |

^{a}Accuracy = (1 − |NMR − grav.|/grav.) × 100
^{b}All error is within 1.5%.
^{c}All error is within 12%.
^{d}All error is within 0.002%.
^{e}All error is within .05%.
^{f}All error is within 0.1%.

TABLE 2
Summary of NMR and gravimetric data obtained
from the human lyophilized plasma sample set

| | NMR time | | | | | prediction | |
|---|---|---|---|---|---|---|---|
| sam- | constants (ms) | | PWC (%) | | | accuracy (%)^{a} | |
| ple | T₂^{b} | T₁^{c} | grav.^{d} | T₂ calc.^{e} | T₁ calc.^{f} | T₂^{g} | T₁^{h} |
| 1 | 71 | 1802 | 70.2 | 74.3 | 69.1 | 94.1 | 98.5 |
| 2 | 80 | 1945 | 72.0 | 75.3 | 71.5 | 95.5 | 99.3 |
| 3 | 98 | 2096 | 74.0 | 77.0 | 74.0 | 96.0 | 100.0 |
| 4 | 98 | 2095 | 75.9 | 77.0 | 74.0 | 98.6 | 97.5 |
| 5 | 140 | 2782 | 78.0 | 79.8 | 83.2 | 97.7 | 93.3 |
| 6 | 184 | 2598 | 79.8 | 82.0 | 81.0 | 97.3 | 98.5 |
| 7 | 211 | 2691 | 81.7 | 83.1 | 82.2 | 98.3 | 99.4 |
| 8 | 258 | 2814 | 83.5 | 84.7 | 83.6 | 98.6 | 99.9 |
| 9 | 304 | 2906 | 85.9 | 86.0 | 84.7 | 99.8 | 98.7 |
| 10 | 385 | 3049 | 87.8 | 87.9 | 86.2 | 99.9 | 98.2 |
| 11 | 455 | 3243 | 89.7 | 89.3 | 88.2 | 99.6 | 98.4 |
| 12 | 603 | 3488 | 91.7 | 91.5 | 90.6 | 99.8 | 98.9 |
| 13 | 800 | 3911 | 95.1 | 93.8 | 94.4 | 98.7 | 99.3 |
| 14 | 1023 | 4200 | 95.7 | 95.7 | 96.7 | 100.0 | 99.0 |
| 15 | 1453 | 4503 | 98.0 | 98.5 | 99.0 | 99.5 | 99.0 |

^{a}Accuracy = (1 − |NMR − grav.|/grav.) × 100
^{b}All error is within 1.4%.
^{c}All error is within 11%.
^{d}All error is within 0.002%.
^{e}All error is within 0.07%.
^{f}All error is within 0.001%.
^{g}All error is within 0.04%.
^{h}All error is within 0.1%.

It is another useful observation that, while the transient relaxation signals obtained in this study could be analyzed with the inverse Laplace transform (ILT) or other multiexponential decomposition signal processing algorithms to reveal multiple T₂ and T₁ components, a stronger correlation could be mapped more easily to PWC from the more simplistic single component exponential fitting with non-linear least squares (NLLS) regression. Previous studies reported in depth analyses of the factors that change the distribution of T₂ in blood (Cistola & Robinson, 83 Trends Anal. Chem. 53 (2016)). However, it is more convenient from a clinical standpoint to pursue the single exponential approach for three reasons. First, a regression-type analysis is more stable than the ILT and less computationally expensive because it is non-iterative. Second, and unlike the ILT, single exponential fitting is readily automated to yield fast and consistent results, which makes it easier to use in a large-scale hospital setting. Finally, a single T₂ or T₁ value from a blood sample can be directly mapped to a standard curve with no ambiguity, which makes the approach attractive from a practical standpoint.

Although one would expect two-to-three exponential decay components for blood plasma, that relate in a physical sense to water, lipid, and protein components, with the largest of these being water, it was found that even in the lowest PWC case analyzed (70%), a mono-exponential fit provided an R2 value of 0.9998 in the worst case. When compared to a three component multi-exponential fit, the R2 value for the same sample was 0.9998. Therefore, without sacrificing the goodness of fit of the CPMG data, least squares fitting can rapidly extract a single decay constant. This constant is essentially a weighted average of the multiple T₂ values described in detail by Cistola and Robinson (83 Trends Anal. Chem. 53 (2016)), that is simply obtained without any of the computational instability introduced by fixed component ILT. A modeling routine can be automated much more readily by fitting to a mono-exponential, as the correlation map is much simpler between a single decay constant and PWC. In practice, this simplicity makes the model less prone to error.

Since the goal of the NMR experiment in this work is to obtain estimates of T₂ and T₁ values, or the time constants of the transient relaxation signals, any data processing that sacrifices some amplitude but offers significant improvements in noise is attractive. All raw time-dependent transient relaxation signals were Fourier transformed and multiplied by a Gaussian peak in the frequency domain. The improved transient signal is then obtained from an inverse Fourier transform. Operation in this way significantly improves the signal-to-noise in both the CPMG and saturation recovery experiments as shown in FIGS. 1-4. The bandwidth of the Gaussian apodization function used to multiply the data in the frequency domain was 100 Hz, a value large enough not to skew the measured T₂ and T₁ values.

### Example 2. Correlation of T₂ and T₁ to PWC

Plots of the gravimetric PWC as a function of T₂ and T₁ value are provided in FIG. 5 and FIG. 6 respectively. The solid squares and diamonds in these plots pertain to the respective pseudoplasma and human lyophilized plasma samples. In both FIG. 5 and FIG. 6, the solid and dashed lines correspond to fits of the measured respective pseudoplasma and human lyophilized plasma data to the function PWC=A log(Tₙ)+B for n=1, 2. More specifically, the variation of gravimetric PWC with NMR T₂ value for the pseudoplasma sample set is shown in FIG. 5 as the solid squares, and a similar plot for the same sample set, where instead the independent variable is the NMR T₁ value, is provided in FIG. 6 as the solid squares. A summary of the A and B values for the two separate time constants and the two separate samples is shown in Table 3. It is clear from these two plots that both the NMR T₂ and T₁ values correlate well with gravimetric PWC for the pseudoplasma sample set. In all fits, the R² value was greater than 0.97.

TABLE 3
Best fit parameters for the shifted log function PWC =
A log(Tₙ) + B for n = 1, 2

| | |
|---|---|
| T₂ | T₁ |

| sample | A^{a} | B^{a} | A^{a} | B^{a} |
|---|---|---|---|---|
| pseudoplasma | 11.61 | 12.66 | 38.02 | −221.50 |
| human lyophilized plasma | 8.00 | 40.29 | 32.66 | −175.78 |

^{a}All error is within 1.8%.

To create models between non-linearly related variables, a log-linear function is typically the first choice, due to its flexibility and generalizability (Bishop, Feinberg, & Holland, Discrete Multivariate Analysis: Theory and Practice (2007); Steyerberg, Clinical Prediction Models: A Practical Approach to Development, Validation, and Updating (2009)). Log-linear models are one of the most prevalent types of statistical models, and they are known by many names, such as Gibbs distributions, undirected graphical models, Markov random fields or conditional random fields, exponential models, and (regularized) maximum entropy models. Logistic regression and Boltzmann machines are special types of log-linear models. Occam's razor, or the principle of parsimony, dictates that the least complex model with the smallest number of parameters to adequately map a relationship between variables is the best choice for a predictive model. This is because overfitting can lead to a loss of generality (Hawkins, 44 J. Chem. Inf. Comput. Sci. 1 (2004)). Despite creating a very good description of training data, overfit models may not generalize well to unknown ‘test’ data, and therefore have poor predictive power.

The solid lines in FIGS. 5 and 6 represent the shifted log function calculated from the appropriate parameters in Table 3. The parameterized log function allows a PWC to be calculated from the NMR relaxation time constant value. Such NMR estimates of PWC from T₂ and T₁ are also provided in Table 1. The ability of NMR to estimate PWC in this way can be tested by exploring the percent difference between the NMR and gravimetric PWC measurements reported in Table 1. This accuracy is also shown in Table 1. Averages of these T₂ and T₁ respective accuracies of 98.8% and 98.2% suggest that T₂ measurements are slightly better at reproducing gravimetric PWC estimates in the pseudoplasma sample set.

The ability of the NMR T₂ and T₁ values to report the PWC with greater than 98% accuracy simply means that a correlation between T₂, T₁, and PWC has been exploited, signal-to-noise was adequately increased, and a reasonable function that relates T₂ or T₁ values to PWC in an experimentally relevant range was identified. To make this approach useful, a set of human lyophilized plasma samples in the same 70%<PWC<98% range was prepared to serve as a real sample standard. Construction of the gravimetric PWC value versus NMR T₂ and T₁ curves for these samples was then used to determine PWC in porcine and model human blood samples from respective NMR relaxation time values.

The solid diamonds in FIG. 5 and FIG. 6 relate gravimetric PWC to the respective T₂ and T₁ values for the human lyophilized plasma sample set. Like Table 1, Table 2 for the human lyophilized plasma sample set reports these NMR T₂ and T₁ and gravimetric PWC values. The dashed lines in FIG. 5 and FIG. 6 correspond to a shifted log function calculated from the appropriate parameters in Table 3. These parameterized log functions are used to estimate PWC from the NMR T₂ and T₁ values and the results of this calculation are also shown in Table 2. Again, as was accomplished for the pseudoplasma sample set above, the accuracy of the NMR PWC estimate was calculated by comparison to the gravimetric PWC value. A summary of these accuracies for each of the human lyophilized plasma samples is shown in Table 2. Averages of these T₂ and T₁ PWC estimate accuracies of 98.2% and 98.5% suggest that the NMR relaxation time constants faithfully reproduce gravimetric PWC values. Consideration of the T₂ and T₁ data simultaneously using a multidimensional regression does not improve the accuracy. The average accuracy in this mixed situation is midway between the accuracies for the separate one-dimensional cases. To evaluate the repeatability of the NMR testing, each sample was analyzed in triplicate. The standard error between trials is plotted in FIGS. 5 and 6, and was much greater for T₁ measurements than for T₂. In fact, the error in T₂ measurements is smaller than the data markers and is therefore not graphically visible in FIG. 5. This was one factor that suggested that the predictive model be based on T₂, rather than T₁, NMR measurements.

It can be noted in FIGS. 5 and 6 that the T₂ and T₁ values for human lyophilized plasma samples are slightly shorter than those for the pseudoplasma samples at the same PWC. It is possible that the separation of red blood cells from the plasma was not perfect. These residual red blood cells could lyse to release hemoglobin or paramagnetic deoxy-hemoglobin, which would shorten the spin relaxation times in a consistent way across all samples. Another possibility is that the subjects who provided the samples had some trace amounts of free hemoglobin, which was not measurable by spectrophotometry (or by eye), but was enough to impact the T₂ and T₁ values. Blood collection itself could also cause some hemolysis.

### Example 3. Testing of PWC Model

In order to determine whether portable NMR relaxometry can estimate the PWC in real blood samples, the shifted log function with the parameters reported in Table 3 for the human lyophilized plasma data relating T₂ value to gravimetric PWC in FIG. 5 and Table 2 was used. Since the T₂ and T₁ measurements report a PWC value equally well, and because T₂ measurements demonstrated higher repeatability, only T₂ data was obtained for the porcine and model human blood samples. Moreover, the CPMG experiment for estimating T₂ is significantly less time consuming than the saturation recovery pulse sequence used to determine T₁. It should be clear that, since NMR relaxation times can be magnetic field-and temperature-dependent, corresponding fit parameters from a model cannot be employed on NMR systems operating at different static field strengths and temperatures. The model parameters reported here only apply for this specific magnet at the reported 23° C. temperature. To accomplish this work with other magnets or at other temperatures, calibrations like those reported here must be completed.

Table 4 reports the NMR T₂ value for a porcine blood sample set and the PWC value determined from that T₂ value and the human lyophilized plasma parameterized, shifted log function. A gravimetric analysis of these same samples produced the PWC values shown in the fourth column in Table 4. Table 4 also reports the accuracy of the NMR-determined PWC for each sample in reference to the gravimetric PWC value in that same sample. This accuracy is a true representation of the performance of the NMR-based PWC estimation method. The accuracies reported in Tables 1 and 2 communicate self-consistency within each individual model. Here the NMR PWC estimate in porcine blood is based on a gravimetric PWC measurement in human lyophilized plasma via the parameterized, shifted log function determined from human lyophilized plasma. It is this PWC estimate, based on a gravimetric PWC value from human lyophilized plasma, that is compared to the gravimetric PWC measurement for porcine blood in Table 4. The 98.7% average prediction accuracy over all samples shown in Table 4 is surprisingly as good as the self-consistency checks for all of the relaxation models considered in Tables 1 and 2.

TABLE 4
Summary of NMR and gravimetric data obtained
from the porcine blood sample set

| | | |
|---|---|---|
| T₂ | PWC (%) | prediction |

| sample | (ms)^{a} | NMR^{b} | grav.^{c} | accuracy (%)^{d} |
|---|---|---|---|---|
| 1 | 554 | 90.8 | 91.6 | 99.2 |
| 2 | 451 | 89.2 | 91.3 | 97.7 |
| 3 | 434 | 88.9 | 90.6 | 98.1 |
| 4 | 406 | 88.3 | 90.1 | 98.0 |
| 5 | 372 | 87.6 | 89.2 | 98.2 |
| 6 | 399 | 88.2 | 88.9 | 99.3 |
| 7 | 303 | 86.0 | 86.8 | 99.1 |
| 8 | 259 | 84.8 | 85.1 | 99.6 |
| 9 | 248 | 84.4 | 85.0 | 99.3 |

^{a}All error is within 2.8%.
^{b}All error is within 0.006%.
^{c}All error is within 0.002%.
^{d}Accuracy = (1 − |NMR − grav.|/grav.) × 100. All error is within 1.7%.

### Example 4. Correction of Electrolyte Test

The real value in rapid, non-destructive PWC estimates is in improving clinical measurements. One such measurement is the clinical monitoring of electrolyte concentrations in blood using both D-ISE and I-ISE based devices. It is well known that electrolyte concentrations derived from D-ISE and I-ISE as [D-ISE] and [I-ISE] respectively, differ by a scaling factor as [D-ISE]=α[I-ISE]. The reason that the two ISE derived concentrations differ is that the algorithm relating the electrochemical response to the electrolyte concentration in the I-ISE device assumes a 93% PWC. As mentioned above, a constant α=1.11 value was proposed by Fogh-Andersen et al. and was ultimately adopted by the IFCC (Fogh-Andersen, Wimberley, Thode, & Siggaard-Andersen, 189 Clin. Chim. Acta 33 (1990); Fogh-Andersen & D'Orazio, 44 Clin. Chem. 655 (1998); D'Orazio et al., 51 Clin. Chem. 1573 (2005)), although there are many cases during critical illness where α≠1.11 and thus I-ISE measurements fail to report accurate blood and blood plasma electrolyte concentrations. In these cases, where the actual PWC differs from 93%, better estimates of a are required.

The calculations summarized in Table 5 examine the consequence of correcting the I-ISE measurement by choosing α=93%/PWC where PWC is the NMR-determined value. Table 5 reports the NMR T₂ values obtained from a model human blood sample set along with the NMR-determined PWC value calculated from the parameterized, shifted log function for the human lyophilized plasma sample set. The laboratory D-ISE and clinical I-ISE estimates for the Na⁺ concentration in these same samples is also reported in Table 5, along with two separate I-ISE corrections where a was calculated using both NMR and gravimetric values for PWC. The accuracies of these two separate corrections were also probed by comparison to the D-ISE Na⁺ concentration values. The results of this exercise are also shown in Table 5. The slightly better 98.1% average accuracy of the NMR based I-ISE Na⁺ concentration correction in comparison to the 97.8% average value for the gravimetric measurement is a useful result.

TABLE 5
Summary of NMR, gravimetric, D-ISE Na⁺ concentration, and I-ISE
Na⁺ concentration results from model human blood samples

| | | | | [I-ISE] | | | prediction | |
|---|---|---|---|---|---|---|---|---|
| sam- | NMR | | [D-ISE] | (mmol/L) | | | accuracy (%)^{a} | |
| ple | T₂(ms)^{b} | PWC(%)^{c} | (mmol/L)^{d} | clinic^{e} | NMR^{f} | grav.^{g} | NMR^{h} | grav.^{h} |
| 1 | 332 | 80.1 | 173.0 | 151 | 175.7 | 175.5 | 98.5 | 98.6 |
| 2 | 329 | 80.0 | 171.4 | 150 | 175.8 | 174.0 | 97.5 | 98.5 |
| 3 | 695 | 88.6 | 149.7 | 141 | 147.5 | 145.5 | 98.5 | 97.2 |
| 4 | 690 | 88.6 | 151.4 | 142 | 148.5 | 146.8 | 98.1 | 97.0 |

^{a}Accuracy = (1 − |[I-ISE] − [D-ISE]|/[D-ISE]) × 100
^{b}All error is within 1.5%
^{c}All error is within 0.004%
^{d}All error is within 0.5%
^{e}All error is within 1%
^{f}All error is within 0.01%
^{g}All error is within 0.002%
^{h}All error is within 0.9%

In summary, the A and B values obtained from a T₂ analysis of the human lyophilized plasma samples were used as a model to relate the T₂ values measured from porcine and model human blood samples to PWC percentages. The PWC percentages obtained in this way are shown in the third column of Tables 4 and 5 for the porcine and model human blood samples respectively. The results demonstrate that when calibrated, a simple, non-destructive, rapid NMR estimate of blood PWC can be used in tandem with clinical I-ISE measurements to faithfully produce equivalent results to the more lengthy and destructive D-ISE tests and sample lyophilization.

Although the foregoing has been described in some detail by way of illustration and example for purposes of clarity and understanding, one of skill in the art will appreciate that certain changes and modifications can be practiced within the scope of the appended claims in view of the foregoing discussion, relevant knowledge in the art, and references discussed above in connection with the Background and Detailed Description, the disclosures of which are all incorporated by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention

1. A method for determining the water content of a body fluid, the method comprising:
analyzing a body fluid sample by portable nuclear magnetic resonance (NMR) relaxometry.

2. The method of claim 1, wherein the analyzing of the body fluid sample by NMR relaxometry comprises:
determining, using NMR relaxometry, a spin-spin relaxation rate constant T_{2,sample} of the body fluid sample and/or a spin-lattice rate constant T_{1,sample} of the body fluid sample; and
calculating the water content of the body fluid sample using the determined T_{2,sample} and/or T_{1,sample}.

3. The method of claim 1, wherein the calculating comprises applying a correlation between water content of the body fluid and one or both of T₂ and T₁.

4. The method of claim 3, wherein the correlation is derived from a measurement of a spin-spin relaxation rate constant T_{2,standard} of a standard solution and/or a spin-lattice rate constant T_{1,standard} of the standard solution, wherein the standard solution has a known standard water content.

5. The method of claim 3, wherein the correlation is derived from measurements of spin-spin relaxation rate constants of two or more standard solutions and/or spin-lattice rate constants of the two or more standard solutions, wherein at least two of the two or more standard solutions have different known standard water contents.

6. The method of claim 4, wherein each known standard water content is between 70% and 98%.

7. The method of claim 3, wherein the correlation comprises one or more log-linear functions.

8. The method of claim 3, wherein the correlation comprises one or more functions each independently having the form:
(water content)=A log(Tₙ)+B, wherein A and B are each independently constants, and wherein n is 1 or 2.

9. The method of claim 4, wherein each known standard water content is determined using gravimetric data.

10. The method of claim 4, wherein at least one standard solution comprises bovine serum albumin, lipid, sodium chloride, and urea.

11. The method of claim 4, wherein at least one standard solution comprises porcine blood or plasma.

12. The method of claim 4, wherein at least one standard solution comprises human blood or plasma.

13. The method of claim 1, wherein the body fluid sample is a blood plasma sample, and wherein the water content of the body fluid sample is a plasma water content (PWC).

14. The method of claim 2, wherein each spin-lattice rate constant is measured by saturation recovery.

15. The method of claim 2, wherein each spin-spin relaxation rate constant and spin-lattice rate constant is determined further using single component exponential fitting with non-linear least squares regression.

16. The method of claim 2, wherein each spin-spin relaxation rate constants and spin-lattice rate constant is determined further using a Fourier transformation, a multiplication by a Gaussian peak, and an inverse Fourier transformation.

17. A method for correcting an electrolyte concentration estimate, the method comprising:
estimating an electrolyte concentration in a sample using an ion selective electrode;
determining the water content of the sample using the method of claim 1; and
correcting the estimated electrolyte concentration using the determined water content.

18. A portable nuclear magnetic resonance (NMR) apparatus for the analysis of water content in body fluids.

19. The portable NMR apparatus of claim 18, having a tank circuit probe comprising a solenoid radiofrequency (RF) coil configured to accept a body fluid sample container, wherein the tank circuit probe is disposed between two sides of an opposing poleface magnet.
